(19) United States
(12) Patent Application Publication
(10) Pub. No.: US 2025/0010048 A1
(43) Pub. Date: Jan. 9, 2025

(54) SURGICAL DILATOR DEVICE

(71) Applicant: LSI Solutions, Inc., Victor, NY (US)

(72) Inventor: Jude S. Sauer, Pittsford, NY (US)

(21) Appl. No.: 18/507,255

(22) Filed: Nov. 13, 2023

Related U.S. Application Data
(60) Provisional application No. 63/424,574, filed on Nov. 11, 2022.

(51) Int. Cl. A61M 29/00; A61B 17/3209; A61B 17/00

(52) U.S. Cl. CPC A61M 29/00; A61B 17/3209; A61B 2017/00367

(57) ABSTRACT

A surgical device includes a distal housing assembly disposed at a distal end of a shaft, the distal housing assembly including a housing portion extending from a proximal end to a distal end along a non-linear housing axis. The distal housing assembly also includes a blade portion having a blade tip that is configured to cut through tissue. The blade portion is coupled to a lever of the surgical device such that (a) when the lever is in a first lever position, the blade tip is in a first blade position in which the blade tip is disposed proximal to the distal end of the housing portion and (b) when the lever is in a second lever position, the blade tip displaces to a second blade position in which the blade tip is disposed distal to the distal end of the housing portion.

### REFERENCE TO RELATED APPLICATIONS

This application claims the benefit of U.S. Provisional Patent Application No. 63/424,574, filed Nov. 11, 2022, which is incorporated by reference herein in its entirety.

### FIELD OF THE INVENTION

The claimed invention relates to surgical devices, and more specifically to a dilator device used in a surgical procedure.

### BACKGROUND

In some medical procedures, it is necessary to perforate a membrane at a precise location. Once such procedure is an aortic valve replacement that involves a bypass and a subsequent restarting of the heart. Following the restarting of the heart, the left ventricle of the heart may initially have an inadequate ability to push blood through the new aortic valve, which can lead to distention of the heart and constriction of the coronary vasculature. To reduce these risks, the left ventricle may be perforated and a cannula may be inserted through the perforation and into the left ventricle (“Left Ventricular Vent Cannulation”), and the blood may be drained from the left ventricle by the cannula prior to restarting the heart. However, due to the buildup of blood in the left ventricle, currently-used cannulation techniques do not allow the surgeon to clearly see the desired area of the left ventricle for perforation. Further, existing devices and techniques do not allow for the creation of specifically sized perforations in a specific location.

### BRIEF DESCRIPTION OF THE DRAWINGS

FIG. 1 is a perspective view of an embodiment of a surgical device;

FIG. 2 is an exploded perspective view of the embodiment of the surgical device of FIG. 1;

FIG. 3A is a left side view of the embodiment of the surgical device of FIG. 1 with the lever in a first lever position;

FIG. 3B is a left side view of the embodiment of the surgical device of FIG. 1 with the lever in a second lever position;

FIG. 4 is a right side view of the embodiment of the surgical device of FIG. 1 with a portion of the main housing omitted for clarity;

FIG. 5 is a cross-sectional view of an embodiment of a distal housing assembly of the surgical device of FIG. 1 taken along section line 5-5 of FIG. 3A;

FIG. 6A is a perspective view of an embodiment of a distal housing assembly of the surgical device of FIG. 1 with the shaft omitted for clarity;

FIG. 6B is an exploded perspective view of the embodiment of the distal housing assembly of FIG. 6A;

FIG. 7A is a top view of the embodiment of the distal housing assembly of FIG. 6A;

FIG. 7B is a side view of the embodiment of the distal housing assembly of FIG. 6A;

FIG. 7C is a bottom view of the embodiment of the distal housing assembly of FIG. 6A;

FIG. 7D is a cross-sectional view of the embodiment of the distal housing assembly of FIG. 6A taken along a shaft axis;

FIG. 8A is a perspective view of the embodiment of the distal housing assembly of FIG. 6A with a blade portion in a second blade position;

FIG. 8B is a perspective view of the embodiment of the distal housing assembly of FIG. 6A with the blade portion in a first blade position;

FIG. 9A is a side view of the embodiment of the distal housing assembly of FIG. 6A with a blade portion in the first blade position;

FIG. 9B is a side view of the embodiment of the distal housing assembly of FIG. 6A with the blade portion in the second blade position;

FIG. 10 is a left side view of the embodiment of the surgical device of FIG. 1 with the shaft and a portion of the main housing omitted for clarity;

FIG. 11 is a detailed view of a proximal end of the blade portion coupled to a distal end of an actuator rod with the shaft omitted for clarity;

FIG. 12 is a detailed perspective view of a connection member coupled to a lever and a proximal end of the actuation lever with a portion of the main housing portion omitted for clarity; and

FIG. 13 is a cross-sectional view of an embodiment of portions of the shaft taken along the shaft axis.

### DETAILED DESCRIPTION OF THE INVENTION

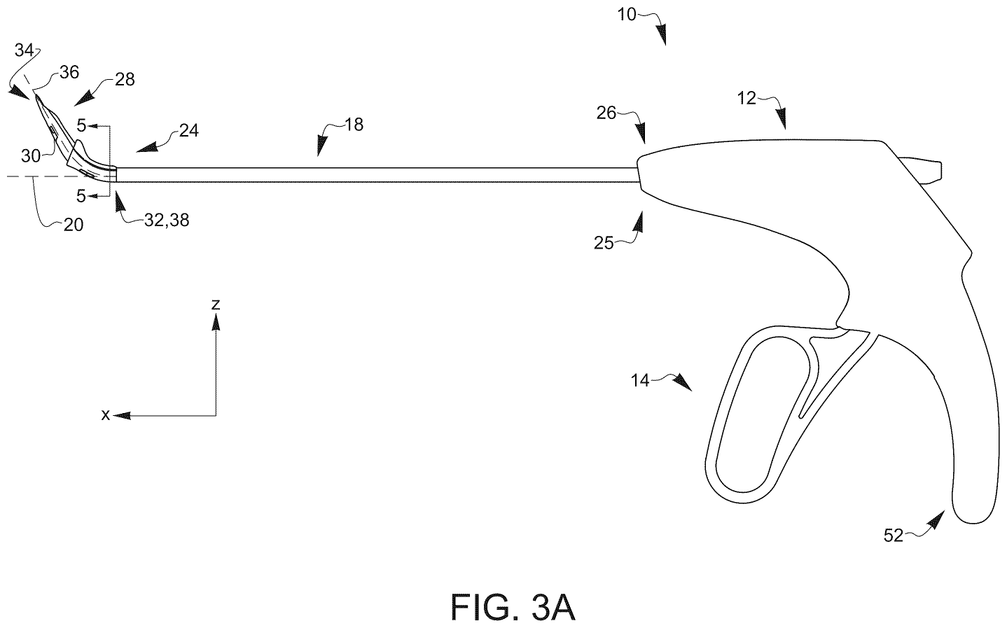

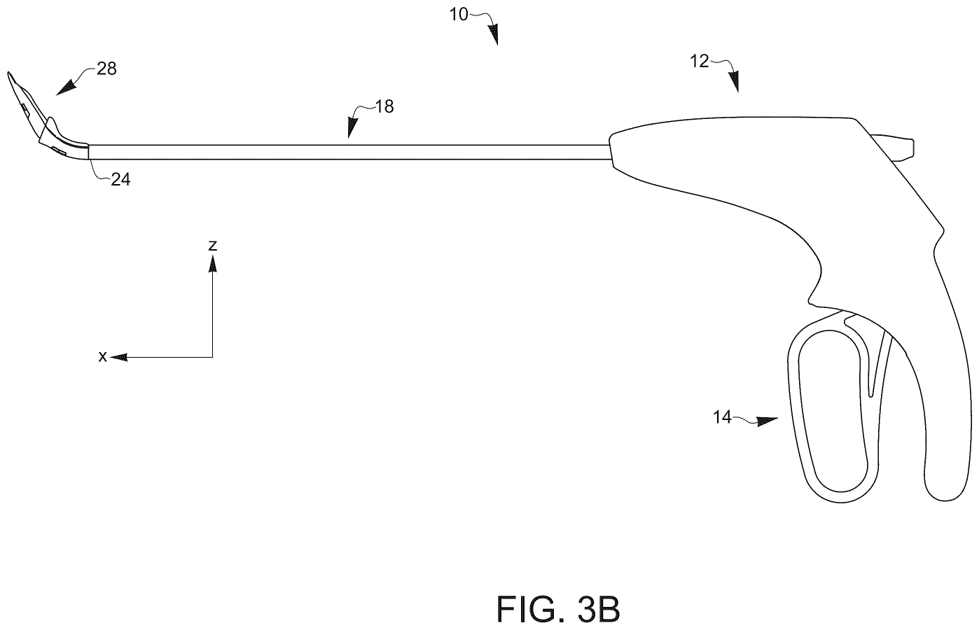

FIG. 3A illustrates a left side view of an embodiment of a surgical device 10 that includes a main housing 12, while FIG. 4 is a right side view of the embodiment of the surgical device 10 with a portion of the main housing 12 omitted for clarity. FIG. 4 illustrates that an actuator, such as a lever 14, is coupled to a first portion 16 of the main housing 12. FIG. 4 also illustrates that a shaft 18 extends along a shaft axis 20 from a proximal end 22 of the shaft 18 to a distal end 24 of the shaft 18. A first portion 25 of the shaft 18 is coupled to a second portion 26 of the main housing 12, and the first portion 25 of the shaft 18 being disposed at or adjacent to the proximal end 22 of the shaft 18.

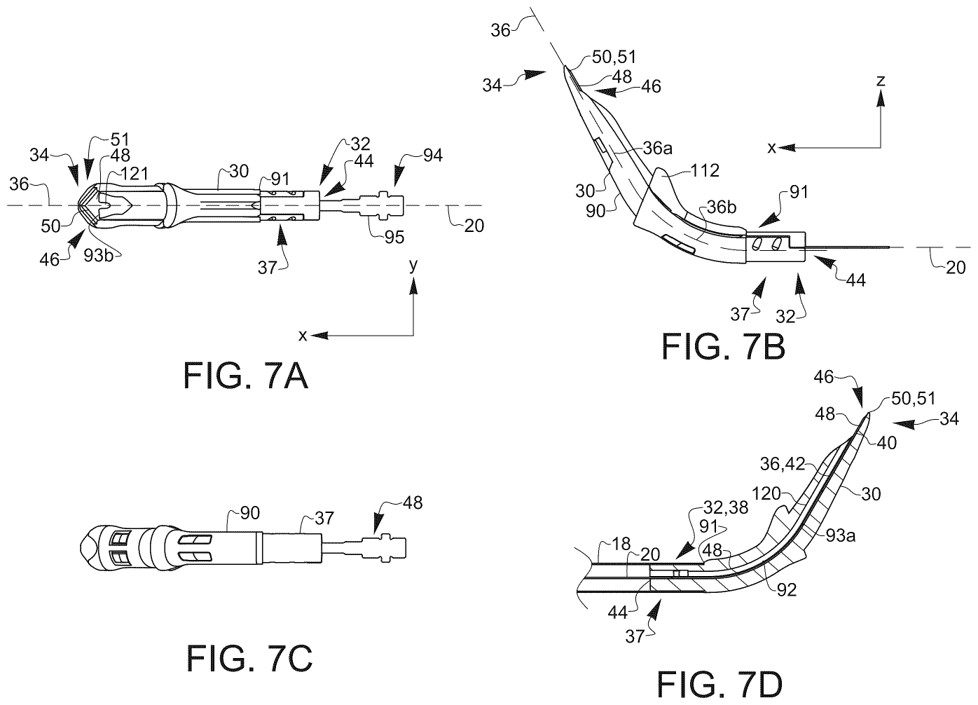

With reference to FIG. 3A, the surgical device 10 further includes a distal housing assembly 28 coupled to the distal end 24 of the shaft 18, and the distal housing assembly 28 includes a housing portion 30 extending from a proximal end 32 to a distal end 34 along a housing axis 36. As illustrated in FIG. 7B, which only illustrates the distal housing assembly 28, at least a portion of the housing axis 36 is non-linear, and a portion 37 of the housing portion 30 that is at or adjacent to the distal end 34 of the housing portion 30 is coupled to a second portion 38 (illustrated in FIG. 3A) of the shaft 18 that is at or adjacent to the distal end 32 of the shaft 18.

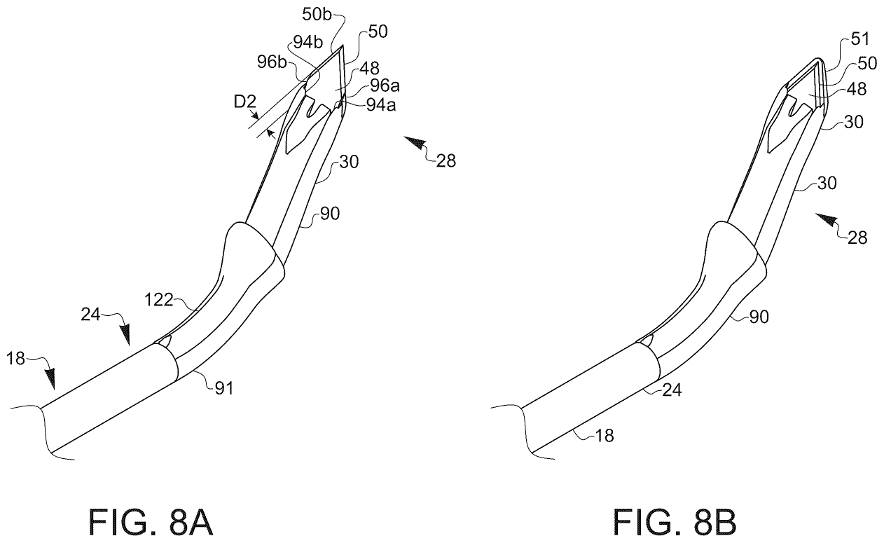

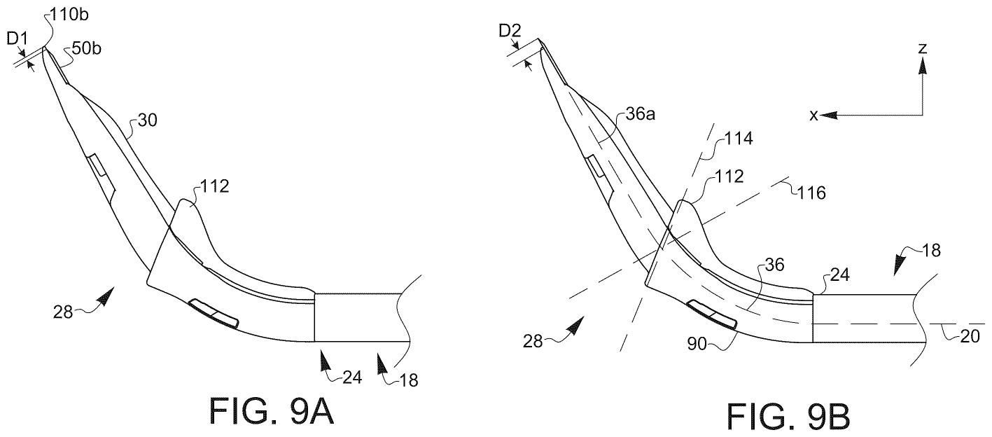

Turning to the cross-sectional view of the distal housing assembly 28 of FIG. 7D, which is taken along a plane that extends along the shaft axis 20 and is parallel to the X-Z plane of the Reference Coordinate System of FIGS. 7A and 7B, a slot portion 40 is at least partially defined by a portion of the housing portion 30, the slot portion 40 extending along a slot axis 42 from a slot proximal end 44 to a slot distal end 46, and the slot axis 42 is aligned with the housing axis 36. The distal housing assembly 28 also includes a blade portion 48 that is at least partially disposed within the slot portion 40, the blade portion 48 having a blade tip 50 configured to cut through tissue, the blade tip 50 being disposed at a distal end 51 of the blade portion 48. The blade portion 48 is coupled to the lever 14 such that when the lever 14 is in a first lever position (illustrated in FIG. 3A), the blade tip 50 is in a first blade position (illustrated in FIGS. 7A, 7B, 8B, and 9A) in which the blade tip 50 is disposed proximal to the distal end 51 of the housing portion 30. However, when the lever 14 is in a second lever position (illustrated in FIG. 3B), the blade tip 50 displaces to a second blade position (illustrated in FIGS. 8A and 9B) in which the blade tip 50 is disposed distal to the distal end 51 of the housing portion 30.

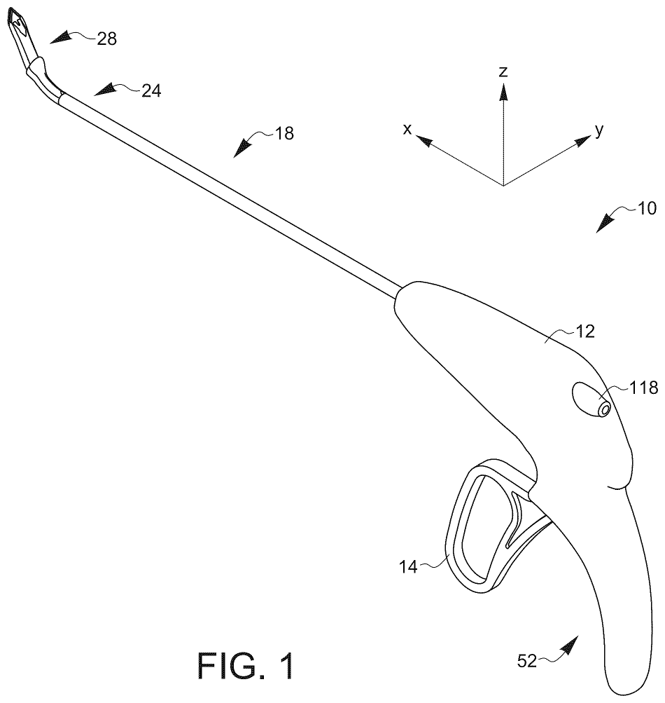

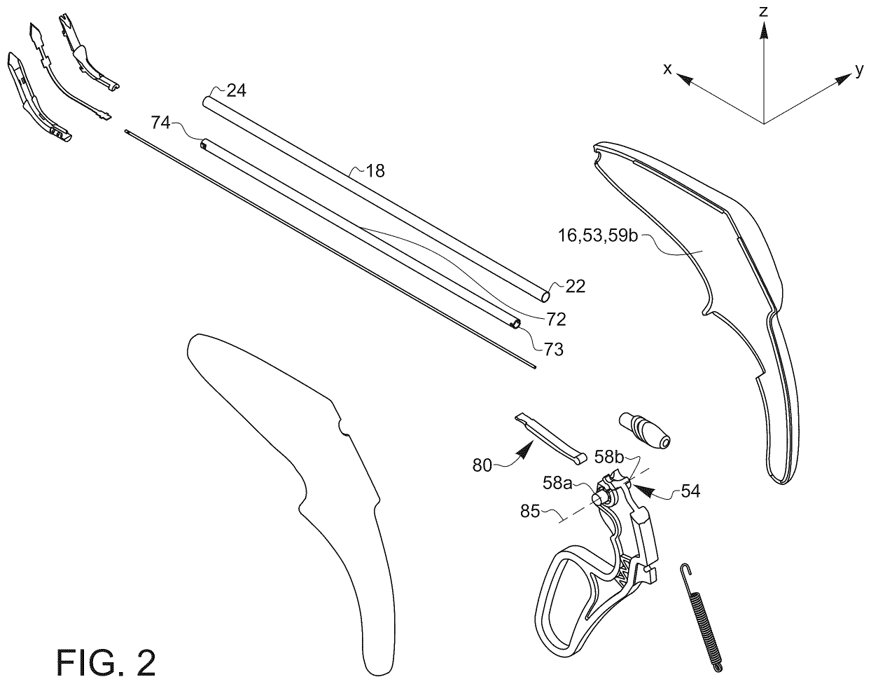

Turning to the surgical device 10 in more detail, FIGS. 1 and 3A illustrate a perspective view and left-side view, respectively, in which the lever 14 is in the first lever position and the blade tip 50 is in the first blade position. The housing 14 includes a grip portion 52 that is adapted to be grasped by a user to engage and displace the lever 14 from the first lever position to the second lever position (illustrated in FIG. 3B) such that the blade tip 50 displaces to a second blade position (illustrated in FIGS. 8A and 9B). Referring to FIG. 4, a first portion 54 of the lever 14 may be rotatably coupled to the main housing 12 at a pivot point 53 at the first portion 16 of the housing such that the lever 14 pivots between the first lever position to the second lever position. The lever 14 may pivot about a pivot axis 85 (see FIG. 2) that extends through the pivot point 53, and the pivot axis 85 may be parallel to the Y-axis of the Reference Coordinate System of FIGS. 1 and 2. As illustrated in the exploded view of FIG. 2, the first portion 54 of the lever 14 may be a pair of aligned bosses 58a, 58b that are received into corresponding cylindrical internal walls 59b (58a not shown) that are each formed on a corresponding interior portion of the housing 14. A first end of a spring 55 may be coupled to a second portion 56 of the lever 14 and a second end of the spring 55 may be coupled to a portion 57 of the interior portion of the housing 14 such that the lever 14 is biased into the first lever position.

As illustrated in FIGS. 1, 2, 3A, 3B, and 4, the surgical device 10 further includes the shaft 18. With specific reference to FIG. 4, the shaft 18 extends along the shaft axis 20 from the proximal end 22 to the distal end 24, and the shaft axis 20 may be aligned with or parallel to the X-axis of the reference coordinate system of FIG. 4. The first portion 25 of the shaft 18, which may be disposed at or adjacent to the proximal end 22 of the shaft 18, may be coupled to the second portion 26 of the main housing 12, and the shaft 18 may be fixed relative to the second portion 26 of the main housing 12. However, in some embodiments, the shaft 18 may rotate relative to the housing 18, or to the second portion 26 of the main housing 12, about the shaft axis 20. A proximal portion 60 of the shaft 18 that extends from the proximal end 22 to an intermediate point 61 may be disposed within the interior portion of the housing 14. Referring to the cross-sectional view of the shaft 18 of FIG. 13, the shaft 18 may be a hollow tube having one or more shaft interior surfaces 64 that define a shaft channel 66 that extends from an open channel proximal end 68 at the proximal end 22 of the shaft 18 to an open channel distal end 70 at the distal end 24 of the shaft 18. The one or more shaft interior surfaces 64 may be a single cylindrical surface having a uniform circular cross-sectional shape extending from the proximal end 22 of the shaft 18 to the distal end 24 of the shaft 18. The elongated shaft 18 and the low-profile distal housing assembly 28 allows the distal housing assembly 28 to be maneuvered through a minimally-invasive incision and through the patient's interior anatomy to a precise location for a perforation procedure, even when blood or other debris obscures the surgeon's direct view of the intended perforation site, thereby minimizing trauma and bleeding in the patient.

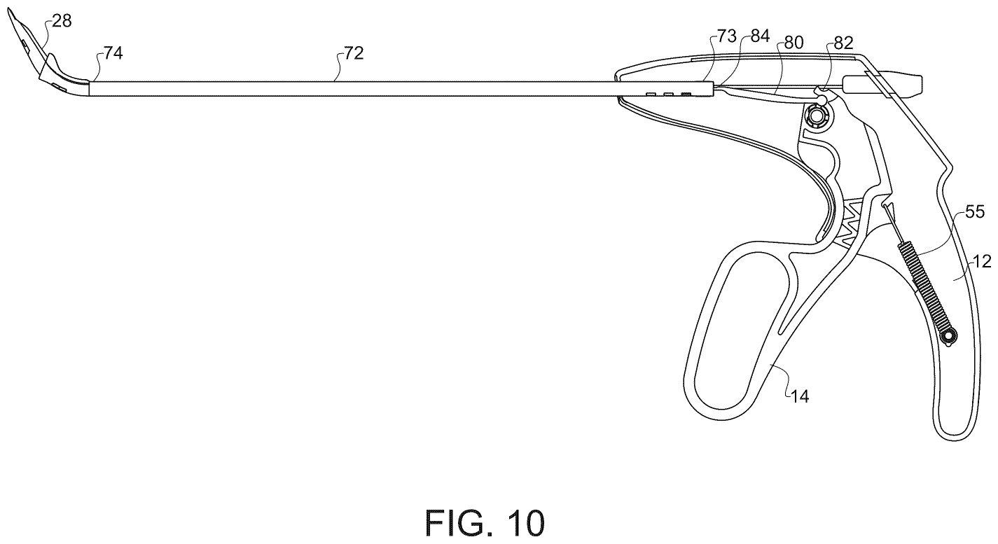

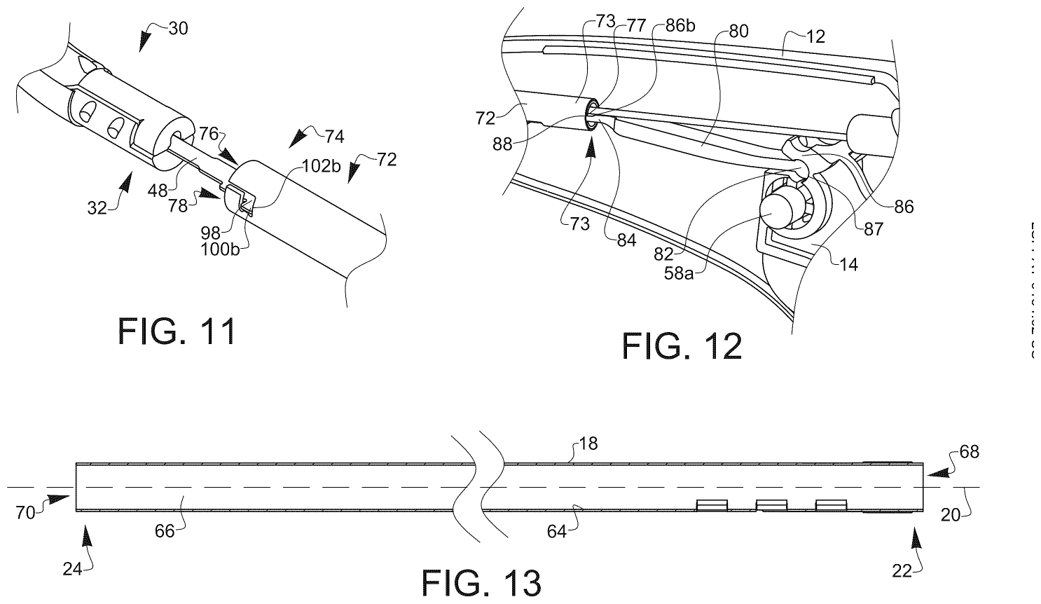

Turning to the exploded view of FIG. 2, the surgical device 10 further includes an actuator rod 72 that extends from a proximal end 73 to a distal end 74 along an axis that is aligned with the shaft axis 20. As illustrated in FIG. 4, the actuator rod 72 is disposed at least partially within the shaft channel 66 such that actuator rod 72 is displaceable relative to the shaft 18 (and the main housing 12) along the shaft axis 20. As illustrated in FIG. 11, the actuator rod 72 may be a hollow tube having one or more rod interior surfaces 75 that define a rod channel 76 that extends from an open channel proximal end 77 (see FIG. 10, with the shaft 18 omitted for clarity) at the proximal end 73 of the actuator rod 72 to an open rod distal end 78 at the distal end 74 of the actuator rod 72. The one or more rod interior surfaces 75 may be a single cylindrical surface having a uniform circular cross-sectional shape extending from the proximal end 73 of the actuator rod 72 to the distal end 74 of the actuator rod 72, and an outer diameter of the actuator rod 72 may be less than a diameter of the shaft channel 66 of the shaft 18 such that the actuator rod 72 may axially displace within the shaft channel 66.

With reference to FIGS. 2, 4, 10, and 12, the surgical device 10 also includes an elongated connection member 80 that extends from a proximal end 82 to a distal end 84. As shown in FIG. 12, the proximal end 82 may be pivotably coupled to a third portion 86 of the lever 14 that may be adjacent to the first portion 54 of the lever 14. The proximal end 82 of the connection member 80 may include a first cylindrical nub 86a that is received into a slot 87 at the third portion 86 of the lever 14, and the connection member 80 may pivot relative to the lever 14 about the first cylindrical nub 86a within the slot 87 about a rotational axis that may be parallel to the Y-axis of the Reference Coordinate System of FIGS. 1 and 2.

The distal end 84 of the connection member 80 may include a second cylindrical nub 86b that is received into a slot 88 formed in the proximal end 73 of the actuator rod 72, and the connection member 80 may pivot relative to the actuator rod 72 about the second cylindrical nub 86b within the slot 88 about a rotational axis that may be parallel to the Y-axis of the Reference Coordinate System of FIGS. 1 and 2. So configured, the actuator rod 72 is coupled to the lever 14 by the connection member 80 such that when the lever 14 is pivoted by a user from the first lever position (illustrated in FIG. 3A) to the second lever position (illustrated in FIG. 3B), the actuator rod 72 is linearly displaced distally along the shaft axis 20 from a first position (illustrated in FIG. 4) in which the distal end 74 of the actuator rod 72 is disposed proximal to (i.e., remote from, or a first distance from) the proximal end 22 of the shaft 18 to a second position in which the distal end 74 of the actuator rod 72 is disposed at or adjacent to (i.e., a second distance that is less than the first distance from) the proximal end 22 of the shaft 18. When the actuator rod 72 displaces distally, the distal end 74 of the actuator rod 72 also displaces distally along the shaft axis 20 in a manner that will be discussed in more detail below. When the lever 14 is released by the user, the spring 55 (see FIG. 4) biases the lever 14 from the second lever position to the first lever position, and the actuator rod 72 is linearly displaced proximally along the shaft axis 20 from the second position to the first position. When the actuator rod 72 displaces proximally, the distal end 74 of the actuator rod 72 also displaces proximally along the shaft axis 20 in a manner that will be discussed in more detail below.

While the illustrated embodiments of the surgical device 10 disclose the actuator as the lever 14, any type of actuator may be coupled to the blade portion 48 in any manner such that when the actuator is displaced from a first actuator position to a second actuator position (and vice versa), the blade tip 50 may displace between the first blade position (illustrated in FIGS. 7A, 7B, 8B, and 9A) and the second blade position (illustrated in FIGS. 8A and 9B) (and vice versa).

Referring to FIGS. 1, 3A, 3B, 8A, and 8B, the surgical device 10 further includes a distal housing assembly 28 disposed at (or coupled to) the distal end 24 of the shaft 18. As illustrated in the isolated view of the distal housing assembly of FIG. 7B (in which the shaft 18 is omitted for clarity), the distal housing assembly 28 includes the housing portion 30 extending from the proximal end 32 to the distal end 34 along the housing axis 36, and at least a portion of the housing axis 36 is non-linear. In particular, a first portion 36a of the housing axis 36 may be linear or substantially linear and may form an acute angle with the shaft axis 20. The acute angle may be any acute angle and may, for example, be between 80 degrees and 15 degrees. In some embodiments, the acute angle of the first portion 36a may be between 70 degrees and 50 degrees. The housing axis 36 may also include a second portion 36b that may be curved, and may have the shape of an arc, segment of a circle, or any other curved shape or combination of curved shapes. The second portion 36b may extend between a proximal end of the first portion 36a and a portion of the shaft axis 20 that is at or adjacent to the distal end 24 of the shaft 20. When viewed along the Z-axis of the Reference Coordinate System of FIGS. 7A and 7B, the housing axis 36 (i.e., both the first portion 36a and the second portion 36b) may be aligned with the shaft axis 20, as illustrated in FIG. 7A, such that the housing axis 20 and the shaft axis 20 are disposed in a plane parallel to the X-Z plane of the Reference Coordinate System of FIGS. 7A and 7B. However, one or both of the first portion 36a and the second portion 36b may not be aligned with the shaft axis 20 and may not be disposed in a plane parallel to the X-Z plane of the Reference Coordinate System of FIGS. 7A and 7B. Further, in some embodiments (not shown), the entire housing axis 36 may be curved or may have a combination of curved shapes.

The housing portion 30 may have an exterior surface 90 that may have any suitable cross-sectional shape or shapes along the housing axis 36 from the proximal end 32 of the housing portion 30 to the distal end 34 of the housing portion 30. For example, all or a portion (or two or more portions) of the exterior surface 90 may have the cross-sectional shape of a circle, oval, polygon, or other closed shape or combination of shapes when viewed along the housing axis 36. Further, the cross-sectional shape may vary along all or a portion (or two or more portions) of the housing axis 36 from the proximal end 32 of the housing portion 30 to the distal end 34 of the housing portion 30. So configured, the housing portion 30 may have a contoured and atraumatic distal end 51 and may have a cross-sectional diameter that gradually increases from the distal end 34 of the housing portion 30 to a point adjacent to the proximal end 34 of the housing portion 30, such as a point adjacent to the distal end 24 of the shaft 18. Such a streamlined shape that gradually increases in thickness allows the housing portion to act as a dilator to increase the size of incisions in tissue.

Turning to FIG. 7B, the portion 37 of the housing portion 30 that extends distally from the proximal end 32 to an intermediate point 91 may have a circular cross-sectional shape that may be configured to be inserted into the distal end 32 of the shaft 18. The portion 37 of the housing portion 30 (or any portion of the housing portion 30) may be coupled to the distal end 32 of the shaft 18 (or any portion of the shaft 18) in any suitable manner. For example, the portion 37 of the housing portion 30 may be sized to frictionally-engage the second portion 38 (illustrated in FIG. 3A) of the shaft 18 that is at or adjacent to the distal end 32 of the shaft 18, and the second portion 38 may include a portion of the shaft interior surface 64 at or adjacent to the distal end 32 of the shaft 18.

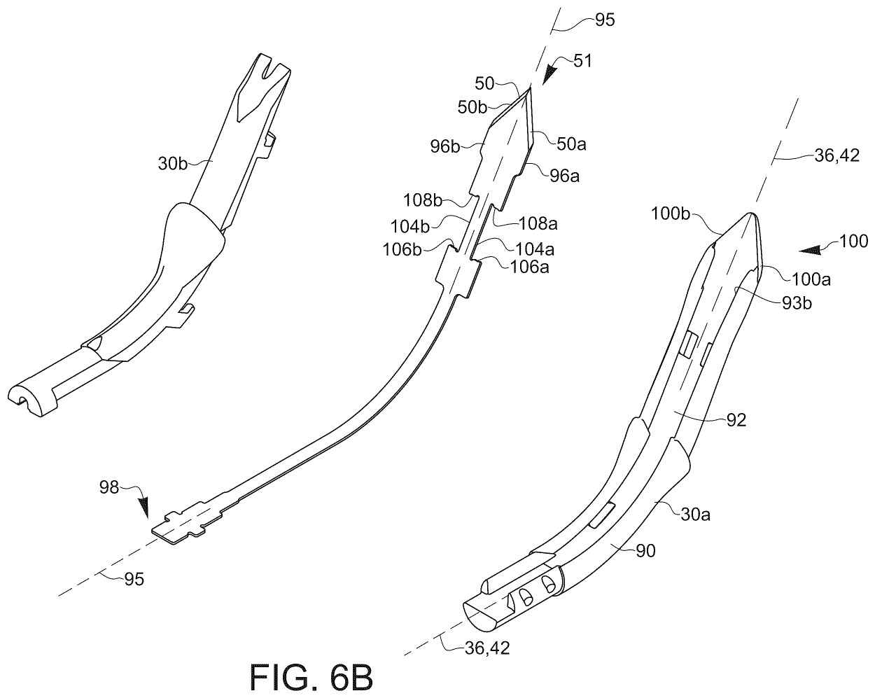

With reference to FIG. 6B, the housing portion 30 may be comprised on a two part assembly, a first housing portion 30a, which includes the contoured distal end 51 of the housing portion 30 and a second housing portion 30a that is secured to the first housing portion 30a to form the housing portion 30.

Referring to FIG. 7D, the distal housing assembly 28 also includes the slot portion 40 that extends through the housing portion 30 a portion along a slot axis 42 from a slot proximal end 44 (that is at or adjacent to the proximal end 32 of the housing portion 30) to a slot distal end 46 (that is at or adjacent to the distal end 34 of the housing portion 30). The slot axis 42 may be aligned with, or parallel to, the housing axis 36. The slot portion 40 may be formed, defined, or configured in any suitable manner to guide the blade portion 48 as the blade tip 50 is displaced from the first blade position (illustrated in FIGS. 7A, 7B, 8B, and 9A) to the second blade position (illustrated in FIGS. 8A and 9B) and vice versa. For example, a first portion 93a of the slot portion 40 may be defined by one or more internal surfaces 92 (see FIG. 6B) of the housing portion 30 that cooperate to form a channel having a rectangular cross-sectional shape when viewed normal the slot axis 42, and the rectangular cross-sectional shape may closely correspond in shape and dimensions to a cross-sectional shape of the widest portion of the blade portion 48 such that the blade portion 48 may displace only along the slot axis 48.

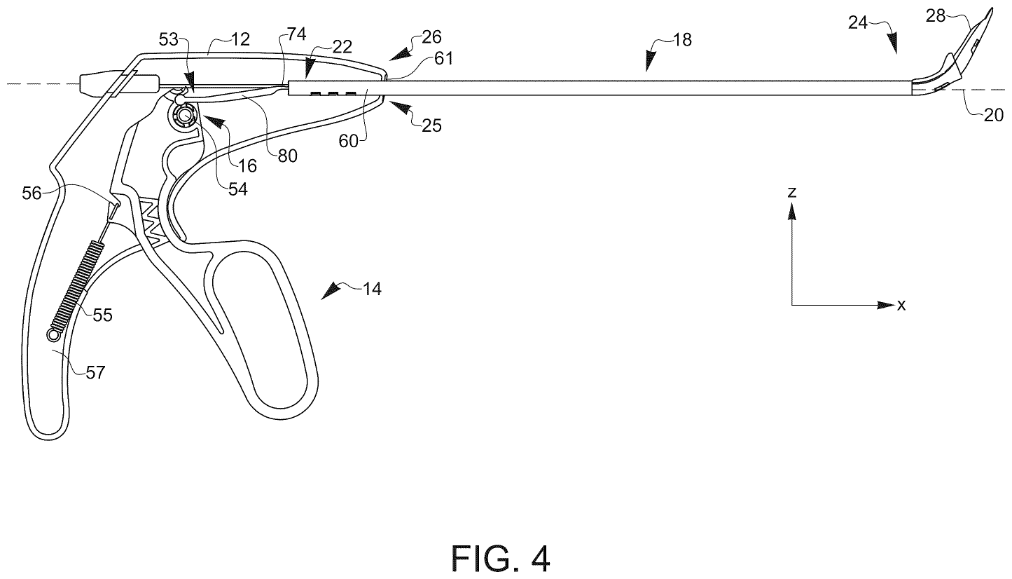

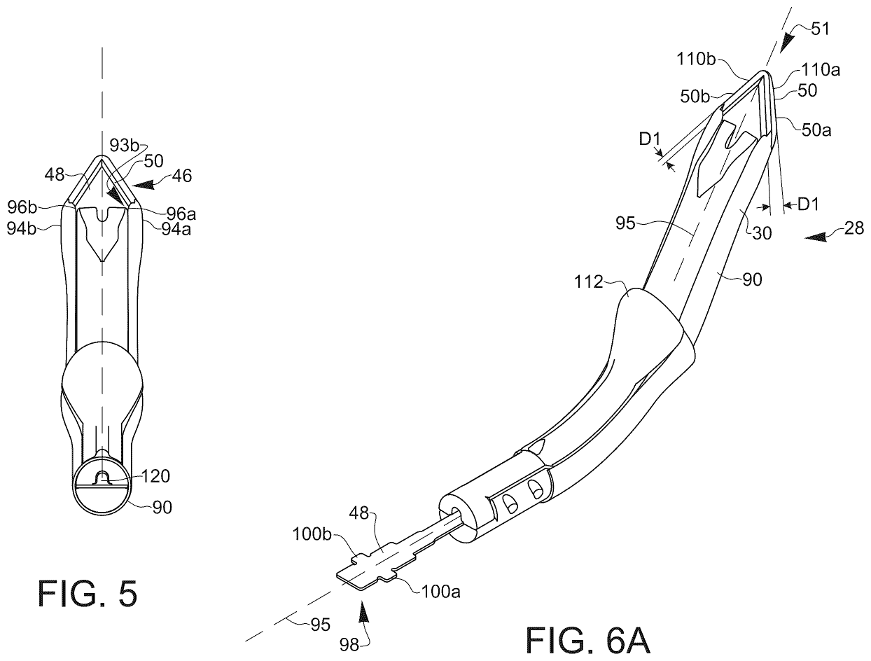

With reference to FIGS. 5, 7A and 8A, the slot portion 40 may also include a second portion 93b that may extend from a distal end of the first portion 93a to the distal end of the slot distal end 46. The second portion 93b may include two lateral edge portions 94a, 94b that each includes a recess adapted to receive a corresponding lateral edge portion 96a, 96b of the blade portion 48 such that the blade portion 48 may displace only along the slot axis 48 within the second portion 93b of the slot portion 40.

Referring to FIGS. 5 to 9B, the distal housing assembly 28 may further include the blade portion 48 that is at least partially disposed within the slot portion 40. As illustrated in the exploded view of the distal housing assembly 28 of FIG. 6B, the blade portion 48 may be elongated and may extend along a blade axis 95 from the distal end 51 to a proximal end 98. The proximal end 98 of the blade portion 48 may be coupled to the distal end 74 of the actuator rod 72 in any suitable manner. For example, the proximal end 98 of the blade portion 48 may include a pair of tabs 100a, 100b (illustrated in FIG. 6A) that may be adapted to be inserted into corresponding tab slots 102a. 102b (with only 102b being illustrated in FIG. 11) formed in the distal end 74 of the actuator rod 72. So configured, when the actuator rod 72 displaces distally (due to the lever 14 displacing from the first lever position illustrated in FIG. 3A to the second lever position illustrated in FIG. 3B), the blade portion 48 also displaces distally (i.e., the blade 48 is displaced along the blade axis and/or the slot axis 42 such that the proximal end 98 is displaced towards the distal end 34 of the housing portion 30) until the blade tip 50 is in the second blade position (illustrated in FIGS. 8A and 9B). The coupling of the proximal end 98 of the blade portion 48 to the distal end 74 of the actuator rod 72 also results in the blade portion 48 displacing proximally (i.e., the blade 48 is displaced along the blade axis and/or the slot axis 42 such that the proximal end 98 is displaced away from the distal end 34 of the housing portion 30) until the blade tip 50 is in the first blade position (illustrated in FIGS. 7A, 7B, 8B, and 9A).

With reference to FIG. 6B, the blade portion 48 may include a pair of limiting slots 104a, 104b formed on opposing lateral edges of the blade portion 48. Each of the limiting slots 104a, 104b may receive a corresponding post (not shown) fixed in the housing portion 30, and each post may contact a proximal edge 106a, 106b of the corresponding limiting slot 104a, 104b to limit distal displacement of the blade portion 48 relative to the housing portion 30. Similarly, each post may contact a distal edge 108a, 108b of the corresponding limiting slot 104a, 104b to limit proximal displacement of the blade portion 48 relative to the housing portion 30.

The blade tip 50 may have any suitable shape to pierce a desired tissue. For example, as illustrated in FIG. 6B, the blade tip 50 may have a V-shape when viewed normal to the blade axis 95, and the blade tip may include a first edge 50a that forms an acute angle with the blade axis 95 and a second edge 50b that forms an acute angle with the blade axis 95. For example, the second edge 50b may form the same acute angle with the blade axis 95 as the acute angle formed by the first edge 50a and the blade axis 95. A leading edge portion 110 of the housing portion 30 that is disposed at the distal end 34 may have a shape that corresponds to the shape of the blade tip 50. For example, the leading edge portion 110 of the housing portion 30 may include a first housing edge 110a and a second housing edge 110b. The first housing edge 110a may correspond in shape and orientation to the first edge 50a of the blade tip 50 and may form an acute angle with the housing axis 36 that is similar to or identical to the acute angle formed by the first edge 50a and the blade axis 95. Similarly, the second housing edge 110b may correspond in shape and orientation to the second edge 50b of the blade tip 50 and may form an acute angle with the housing axis 36 that is similar to or identical to the acute angle formed by the second edge 50b and the blade axis 95.

So configured, when the blade tip 50 is in the first blade position (illustrated in FIG. 6A) in which the blade tip 50 is disposed proximal to the distal end 51 of the housing portion 30, the first edge 50a of the blade tip 50 is parallel to and proximally offset from the first housing edge 110a and the second edge 50b of the blade tip 50 is parallel to and proximally offset from the second housing edge 110b. The first edge 50a of the blade tip 50 may be adjacent to the first housing edge 110a or may be disposed a uniform distance D1 (see FIGS. 6A and 9A) from the first housing edge 110a, and the second edge 50b of the blade tip 50 may be adjacent to the second housing edge 110b or may be disposed the uniform distance D1 (see FIGS. 6A and 9A) from the second housing edge 110b. Accordingly, a contoured shape of the distal end 51 of the housing portion 30, which includes the streamlined and atraumatic tip formed by the first housing edge 110a and the second housing edge 110b, may be advanced through a cavity (using a guide wire, for example) while the blade tip 50 is safely retracted from the distal end 51 of the housing portion 30 to avoid inadvertent lacerations to tissue.

In addition, when the contoured distal end 51 of the housing portion 30 is advanced to a treatment area in which the user desires to pierce tissue, the user pivots the lever 14 from the first lever position to the second lever position such that the blade tip 50 is displaced the second blade position (illustrated in FIGS. 8A and 9B). In this second blade position, the blade tip 50 is advanced distal to the distal end 51 of the housing portion 30 such that the first edge 50a of the blade tip 50 is parallel to and distally offset from the first housing edge 110a and the second edge 50b of the blade tip 50 is parallel to and distally offset from the second housing edge 110b. The first edge 50a of the blade tip 50 may be disposed a uniform distance D2 (see FIGS. 8A and 9B) from the first housing edge 110a and the second edge 50a of the blade tip 50 may be disposed the uniform distance D2 (see FIGS. 8A and 9B) from the second housing edge 110b. Thus, due to the streamlined and low-profile shape of housing portion 30, the user may precisely position the distal end 51 of the housing portion 30 in a confined space and accurately pierce a portion of tissue a desired depth to avoid damaging tissue adjacent to the pierced tissue.

Referring to FIGS. 6A, and 9B, the distal housing assembly 28 further includes a projection portion 112 that extends along a portion of the exterior surface 90 of the housing portion 30. The projection portion 12 may be an annular ring that extends along the portion of an exterior surface 90 of the housing portion 30, and the annular ring may have a uniform cross-sectional shape, such as that of a semi-circle or a parabola, along the entire circumference of the annular ring. The annular ring may be disposed within a projection plane 114 (see FIG. 9B) that intersects the housing axis 36 (and/or the shaft axis 20) but is not normal to the housing axis 36 (or the shaft axis 20). For example, when viewed along the Y-axis of the Reference Coordinate System of FIG. 9B, the projection place may form an angle, such as an acute angle, with a reference plane 116 that is normal to the housing axis 36, and the angle may be between 10 degrees and 70 degrees, or between 20 and 50 degrees, or between 20 degrees and 45 degrees. Both the reference plane 116 and the projection place 114 may intersect the housing axis 36 at the same point.

Referring to FIG. 8A, the distal housing assembly 28 may further include an elongated ridge portion 122 that may extend along a portion of the exterior surface 90 of the housing portion 30, and the ridge portion 122 may extend parallel to the housing axis 36 between the projection portion 112 and a point at or adjacent to the intermediate point 91 disposed adjacent to the distal end 24 of the shaft 18. The ridge portion 122 may have a uniform cross-sectional shape, such as that of a semi-circle or a parabola, that may not extend from the exterior surface 90 as far as the projection portion 112.

So configured, a surgeon may position the distal end 51 of the housing portion 30 of the surgical device 10 into an incision that the surgeon wishes to dilate and the surgeon may proceed to advance the contoured housing portion 30 into the incision. Due to the increasing cross-sectional shape of the exterior surface 90 from the distal end 34 to the proximal end 32, the incision is gradually dilated. When the projection portion 112 is to be inserted through the incision, the surgical device 10 may be rotated as the housing portion 30 is further advanced through the incision such that the projection portion 112, due to the increased cross-sectional area at that location, further dilates the incision. As the housing portion 30 continues to be advanced, the ridge portion 122 may maintain the diameter of the dilated incision.

The surgical device 10 may be guided towards a specific treatment area using a guide wire that is placed in a patient in a known manner. The guide wire (not shown) may be inserted into an open end portion of a guide wire funnel 118 (illustrated in FIG. 1) that is secured to a portion of the main housing 12. The guide wire may then be advanced through the interior of the main housing 12 and into and through the rod channel 76. The guide wire may then enter a guide wire channel 120 (see FIGS. 5 and 7D) disposed within the housing portion 30 of the distal housing assembly 28, and the guide wire may extend from the housing portion 30 at a distal aperture 121 (see FIG. 7A) of the guide wire channel 120.

Various advantages of a surgical device have been discussed above. Embodiments discussed herein have been described by way of example in this specification. It will be apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. The drawings included herein are not necessarily drawn to scale. Additionally, the recited order of processing elements or sequences, or the use of numbers, letters, or other designations therefore, is not intended to limit the claims to any order, except as may be specified in the claims. Accordingly, the invention is limited only by the following claims and equivalents thereto.

What is claimed is:

1. A surgical device comprising:
a main housing;
an actuator coupled to a first portion of the main housing;
a shaft extending along a shaft axis from a proximal end to a distal end, wherein a first portion of the shaft is coupled to a second portion of the main housing;
a distal housing assembly disposed at the distal end of the shaft, the distal housing assembly comprising:
a housing portion extending from a proximal end to a distal end along a housing axis, wherein at least a portion of the housing axis is non-linear, and wherein a portion of the housing portion that is at or adjacent to the proximal end of the housing portion is coupled to a second portion of the shaft that is at or adjacent to the distal end of the shaft;
a slot portion at least partially defined by a portion of the housing portion, the slot portion extending along a slot axis from a slot proximal end to a slot distal end, wherein the slot axis is aligned with the housing axis, and
a blade portion having a blade tip configured to cut through tissue, the blade tip being disposed at a distal end of the blade portion, wherein at least a portion of the blade portion is disposed within the slot portion,
wherein the blade portion is coupled to the actuator such that (a) when the actuator is in a first actuator position, the blade tip is in a first blade position in which the blade tip is disposed proximal to the distal end of the housing portion and (b) when the actuator is in a second actuator position, the blade tip displaces to a second blade position in which the blade tip is disposed distal to the distal end of the housing portion.

2. The surgical device of claim 1, the distal housing assembly further comprising a projection portion that extends along a portion of an outer surface of the housing portion.

3. The surgical device of claim 2, wherein the projection portion is an annular ring that extends along the portion of an outer surface of the housing portion, wherein the annular ring is disposed within a projection plane that intersects the housing axis but is not normal to the housing axis.

4. The surgical device of claim 3, wherein the projection plane makes an acute angle with a reference plane that is normal to the housing axis and that intersections the housing axis at a point in which the projection plane intersects the housing axis.

5. The surgical device of claim 3, wherein the projection plane is not normal to the shaft axis.

6. The surgical device of claim 1, wherein the housing portion includes a first housing portion and a second housing portion coupled to the first housing portion, wherein the slot portion is at least partially defined by a portion of the second housing portion and a portion of the first housing portion.

7. The surgical device of claim 1, wherein the shaft axis is linear.

8. The surgical device of claim 1, wherein at least a portion of the housing axis has the shape of an arc.

9. The surgical device of claim 1, wherein the blade tip has a V-shape that is symmetrical about the housing axis.

10. The surgical device of claim 3, wherein the actuator is a lever pivotably coupled to the first portion of the main housing, the blade portion being coupled to the lever such that (a) when the lever is in a first lever position, the blade tip is in the first blade position in which the blade tip is disposed proximal to the distal end of the housing portion and (b) when the lever is in a second lever position, the blade tip displaces to the second blade position in which the blade tip is disposed distal to the distal end of the housing portion.

11. A distal housing assembly configured to be disposed at a distal end of a shaft of a surgical device, the distal housing assembly comprising:
a housing portion extending from a proximal end to a distal end along a housing axis, wherein at least a portion of the housing axis is non-linear, and wherein a portion of the housing portion that is at or adjacent to the proximal end of the housing portion is configured to be coupled to a portion of the shaft;
a slot portion at least partially defined by a portion of the housing portion, the slot portion extending along a slot axis from a slot proximal end to a slot distal end, wherein the slot axis is aligned with the housing axis, and
a blade portion having a blade tip configured to cut through tissue, the blade tip being disposed at a distal end of the blade portion, wherein at least a portion of the blade portion is disposed within the slot portion,
the blade portion being configured to be coupled to an actuator of the surgical device such that (a) when the actuator is in a first actuator position, the blade tip is in a first blade position in which the blade tip is disposed proximal to the distal end of the housing portion and (b) when the actuator is in a second actuator position, the blade tip displaces to a second blade position in which the blade tip is disposed distal to the distal end of the housing portion.

12. The distal housing assembly of claim 11, further comprising a projection portion that extends along a portion of an outer surface of the housing portion.

13. The distal housing assembly of claim 12, wherein the projection portion is an annular ring that extends along the portion of an outer surface of the housing portion, wherein the annular ring is disposed within a projection plane that intersects the housing axis but is not normal to the housing axis.

14. The distal housing assembly of claim 13, wherein the projection plane makes an acute angle with a reference plane that is normal to the housing axis and that intersections the housing axis at a point in which the projection plane intersects the housing axis.

15. The distal housing assembly of claim 13, wherein the projection plane is not normal to the shaft axis.

16. The distal housing assembly of claim 11, wherein at least a portion of the housing axis has the shape of an arc.

17. The distal housing assembly of claim 11, wherein the blade tip has a V-shape that is symmetrical about the housing axis.

18. The distal housing assembly of claim 11, wherein the blade portion comprises a flexible material.

19. The surgical device of claim 1, wherein the blade portion comprises a flexible material.
